(19) United States
(12) Patent Application Publication
(10) Pub. No.: US 2005/0033311 A1
(43) Pub. Date: Feb. 10, 2005

(54) Medical device comprising a braided portion

(76) Inventors: Signe Uhre Guldfeldt, Hillerod (DK); Egon Triel, Gilleleje (DK); Robert Snell, Suffolk (GB); Sam Martin, Suffolk (GB); Martin Cable, Cambridgeshire (GB); Eleanore King, Northants (GB); Sinan Kiamil, Bedfordshire (GB)

Correspondence Address: JACOBSON HOLMAN PLLC, 400 SEVENTH STREET N.W., SUITE 600, WASHINGTON, DC, 20004, US

(21) Appl. No.: 10/870,477

(22) Filed: Jun. 18, 2004

Related U.S. Application Data
(60) Provisional application No. 60/480,978, filed on Jun. 24, 2003.
(60) Provisional application No. 60/482,140, filed on Jun. 24, 2003.

(30) Foreign Application Priority Data
Jun. 20, 2003 (GB) 0314503.4
Jun. 20, 2003 (GB) 0314504.2
May 19, 2004 (DK) PA 2004 00806

(51) Int. Cl. A61F 11/00

(52) U.S. Cl. 606/108

(57) ABSTRACT

The invention provides a medical device such as a catheter having in a proximal end a braided portion being operable between two configurations, one for insertion of the device into the body and one for retaining the catheter in the body. To reduce ingrowth of body tissue, the braided portion comprises two parts, a first part forming paths for a body liquid, e.g. urine, to be drained from the body through the device to a place of disposal, and a second part having a structure which counteracts cell adhesion better than the first part. During use, the first part is drawn into the second part whereby the cells are separated from first part by the second part and therefore ingrowth of body tissue is reduced or avoided completely. The invention further provides a method of making a device with a braided portion and being improved with respect to ingrowth of body tissue.

The present invention relates to a medical device such as a catheter, and to a method of making such a device. The device comprises a braided portion which can be reshaped e.g. to anchor the device in the body of a living being.

### BACKGROUND OF THE INVENTION

A catheter to be used for draining fluids and having means for retaining the catheter in the body for a period of time is known. One example is the so called Foley-type catheter for urinary drainage of a bladder. Foley catheters are provided with an inflatable balloon in an insertable proximal end portion of the catheter. Inflated, the balloon prevents the catheter from sliding out of urethra. Analogously, tracheal tubes with inflatable balloons for fixation in the trachea during ventilation of a patient exist.

More advanced catheters with a braided catheter section are employed e.g. for urinary drainage of a bladder or for vessel dilation. The braided section forms a meshed structure which, by manipulation of one part relative to another part, can change shape. Such catheters are disclosed e.g. in U.S. Pat. No. 5,041,093 wherein a catheter is made from en elongated flexible tubular member with a conduit. The catheter disclosed therein comprises an axially and radially elastically extensible, woven tube. The woven tube is translatable between three configurations, i.e. a relaxed configuration, an extended configuration and an over-centre configuration. In the over-centre configuration, the woven tube forms a cup-like shape whereby the catheter can be maintained in place inside the body. Braided catheters for insertion and fixation in a human body are also disclosed in U.S. Pat. No. 6,033,413 and in U.S. Pat. No. 4,572,186.

The catheters having a braided, expandable, section are in some applications superior to the traditional Foley-type catheters. It has, however, been found that the braided, and thus partly open or “meshed” tube structure may cause problems in relation to ingrowth of body tissue into the meshes. Polypoid cystitis, or papilliary hyperplasia is a proliferation of the cells causing finger like projections to grow out from the mucosa. Polypoid cystitis can arise even if there is no mesh, eg with a smooth balloon of a Foley and so is not linked to the mesh structure. During indwelling use of a meshed device e.g. for several days, problems, however, may arise if the projections grow through the meshes and get entwined in the filaments of the braid. As a result, it can be difficult to remove the device from the body, and the patient may even experience trauma.

### DESCRIPTION OF THE INVENTION

It is an object of the invention to provide an improvement over the known medical devices. Accordingly, the invention, in a first aspect, provides a medical device comprising an elongated tube with a proximal end for insertion into a body of a living being and an opposite distal end, the tube further comprising a conduit extending in an axial direction, the tube comprising a braided portion e.g. with crossed filaments which form a braiding angle with the axial direction, the braided portion being operable between a first configuration and a second configuration wherein the braided portion is expanded to a larger size in a direction transverse to the axial direction in the second configuration than in the first configuration. In particular, the invention provides a device wherein passages between an outer surface of the device and the conduit are defined e.g. between the filaments, and wherein the device further comprises reduction means for reducing the cross sections of at least a part of the passages. The reduction means could e.g. be constituted by a filler material disposed between the filaments or by a lining, i.e. e.g. a skirt shaped foil which covers at least a part of the braided portion, e.g. in contact with an inner surface of the braided portion or in contact with an outer surface of the braided portion. The filler and the lining can either substitute each other to prevent ingrowth into the braided portion or the filler may be combined with a lining.

The filler could comprise a matrix material disposed between the filaments or partly covering the filaments. The material could be any medical grade polymer that can be dissolved in a solvent or manufactured as a polymer emulsion. Examples of theses are polyurethane, polyurethane dispersions, acrylic, PVC, block copolymers (SIS SBS) etc, natural rubber, silicone, neoprene, nitrile or compositions thereof. Polyurethane, acrylic, PVC, block copolymers (SIS SBS) etc, natural rubber, silicone, or EPO or compositions thereof, could be used if the medical device is made by extrusion or injection moulding.

The lining could form a skirt or mantle on the surface of the braiding. The lining could be a pre-formed funnel shaped piece of a thin elastically flexible polymeric film, e.g. made from any medical grade polymer that can be dissolved in a solvent or manufactured as a polymer emulsion, e.g polyurethane, polyurethane dispersions, acrylic, PVC, block copolymers (SIS SBS) etc, natural rubber, silicone, neoprene. The lining could also be made from PU, Evoprene or nitrile and possibly with a surface layer of a hydrogel. The thickness of the film could be in the range of 3-1000 μm. such as 20-200 μm., such as 40-80 μm. In one embodiment, a foam material, e.g. a hydrogel, PEG-PU, or Kraton™ can be used with in a thickness of up to 5 mm.

In order to further increase the degree of expansion or to form a specific shape of the braided portion in the second configuration, a first part of the braided portion may be located inside a second part of the braided portion when the device is in the second configuration. The first and second parts of the braided portion does not have to be structurally separated, but could form one uniform braided portion, and merely the fold arising by the inverting or rolling of one part of the braided portion into another part of the braided portion defines the transition between the first and second parts of the braided portion. In the first configuration, the braided portion could extend un-folded in the axial direction.

The medical device could be adapted e.g. for injection of fluids into the body, or for draining fluids from the body, and as such be shaped as a catheter. In addition, the device could be adapted for stent delivery, e.g. for placing a stent within the prostatic urethra, or in general for draining fluids from a natural or artificial body lumen, for anal insertion or for insertion into the gastrointestinal region, e.g. with the purpose of fixating a camera or surgical instruments inside the body or in general to establish a passage into the body.

The wording “braided portion” includes in general a portion of the medical device which portion is provided with through-going windows, i.e. openings formed from an outer peripheral surface to the conduit and e.g. being symmetrically arranged to form a uniform grid of windows. More specifically, the braided portion may comprise cross-braided filaments, i.e. threads which are braided over and under each other. Preferably, the braiding enables the filaments to slide relative to each other. Alternatively, the filaments are arranged in two separate and parallel layers wherein the filaments of one of the layers extend in a direction different from the direction of the filaments of the other layer. In each intersection between a filament of one of the layers and a filament of another layer, the filaments of the two layers are joined by adhesion. The braided portion could also be constituted by a section of the device with openings forming a mesh-pattern. Irrespective of the type of braiding, the angle, α, which the filaments form with the axial direction, is important for determining the degree of radial expansion and the more precise shape of the funnel which arises when the first part is displaced into the second part of the braided portion. This is described in further details later, but in general, the braiding may in the second configuration provide an ovoid shape of a larger dimension transverse to the axial direction, a funnel shape, a tulip-like shape, a disc-shape, a hemispherical shell, a conical shell, a elliptic parabolic shell or any other cup like shell or any other shapes which support retaining of the medical device in the body. In order to control the shape and size of the braided portion, in particular in the configuration wherein a first part of the braided portion is located inside a second part of the braided portion, the filaments may form at least two different angles with the axial direction for two different locations in the axial direction of the tube. In one embodiment, the angle changes between two values at two locations along the length of the device, and in another embodiment the angle changes continuously along the axial direction. The braiding could be made from filaments e.g. made from polyester, polyamide, polyalkane, polyurethane, PET, PBT, Nylon, PEEK, PE, Glass Fibre, Metal Wire or Acrylic materials or any composition of the mentioned materials. A preferred material would be PET or polyester.

The medical device, especially the braided portion, may be designed with “shape-memory” such that it will automatically move towards a predetermined shape i.e. towards a relaxed state. In a first embodiment the medical device is designed such that the predetermined shape is the first configuration, i.e. the medical device will have a tendency to move towards the first configuration, but may be moved into the second configuration by axial displacement of the first part of the braided portion into a second part of the braided portion. In some embodiments the first configuration is a configuration wherein the largest dimension of a cross-section of the braided portion is equal or less than the largest dimension of a cross-section of the remaining part of the tube.

In a second embodiment the medical device is designed such that the predetermined shape is the second configuration, i.e. the medical device will have a tendency to move towards the second configuration, but may be moved into the first configuration by axial displacement of the first part of the braided portion out of the second part of the braided portion. In some embodiments the second configuration is a configuration wherein the largest dimension of a cross-section of the braided portion is larger than the largest dimension of a cross-section of the remaining part of the tube.

In a third embodiment the medical device is designed such that the predetermined shape when the first and the second braided parts are located inside the remaining part of the medical device coaxially therewith. When the medical device is located in the body, the second part is displaced out of the remaining part of the medical device to form a medical device in the second configuration, i.e. retained in the body. To operate the medical device between the different configurations, a deployment member could be fastened in the proximal end, e.g. to a proximal tip of the medical device, and extend to the distal end to facilitate manipulation of the proximal end from outside the body.

Positioned in the urethra, the medical device is operated between the first configuration and the second configuration to retain the medical device in the bladder. According to the description above, this operation may include that the first part of the braided portion is moved into, or out of the conduit to shift between a medical device with a double layer proximal end portion and a single layer proximal end portion. The double layer proximal end portion may form coaxially extending layers of the braided portion, i.e. an inner layer formed by the first part of the braided portion, and an outer layer formed by the second part of the braided portion. In this configuration, the double layer proximal end portion expands radially and forms a funnel shaped inlet to the conduit. Since the first part is drawn into the conduit, its braided and open structure is covered by the second part of the tube portion. Accordingly, the structure of the first part could have a more open and “porous” structure than the second part, which second part then protects against ingrowth of body tissue. Accordingly, safe and unobstructed removal of the medical device is facilitated for a medical device having a braided portion. As mentioned previously, a deployment member may operate the medical device between the first and second configuration. The deployment member could be constituted by a rod, e.g. made from braided filaments. The rod could be attached to a proximal end of the first part of the braided portion. The rod could extend the full length of the medical device and be exposed in the vicinity of the distal end, i.e. opposite the proximal end of the medical device. By use of the rod, the user can manipulate the proximal end of the medical device inside the body to switch between the first and second configurations. The funnel shaped proximal end of the medical device, which appears in the second configuration, may form a shape and size which depends on the more specific structure of the braided portion.

The mesh openings or “windows” may form paths between the outer surface of the braided portion and the conduit. The windows are formed between the filaments of the mesh and allow e.g. urine or similar body substances to flow into the medical device. It has, however, been found that body tissue is less liable to adhere to a more closed structure than to a more open or “porous” structures, and closed structure, more specifically, reduces the risk of ingrowth of the tissue. For that reason, the second part could therefore form windows which are smaller than corresponding windows in the first part, or the second part may have a completely closed braided structure, i.e. wherein the spaces between the filaments are sealed e.g. by a polymeric filler material, e.g. by a hydrophilic material. In general, the size of the windows of the entire braided section or at least the second part thereof be reduced down to none at all.

In addition to the mesh openings, the medical device may have an additional opening forming path for the body liquid, the additional opening being located in the vicinity of a transition between the braided portion and the remaining part of the medical device. The additional opening could thus be located in a distal part of the second part of the braided portion or in a proximal part of the remaining part of the medical device. The medical device could moreover have a proximal tip provided with openings for draining fluids into the conduit.

It has been found that body tissue is less liable to adhere to a surface which is constantly moving in relation to the tissue. The braided portion, and in particular the second part thereof may therefore have an outer surface, i.e. a surface towards the body tissue when the medical device is inserted into the body lumen, which surface has a low surface friction characteristic compared with other parts of the medical device. To provide the low friction characteristic, medical device, e.g. the lining or the first part or the second part of the braided portion may have a hydrophilic surface, e.g. provided by a hydrophilic coating of the surface. A hydrophilic coating may further reduce irritation of the body tissue, e.g. mucosa. If a hydrophilic coating is applied to the braided portion, the coating may incorporate an anti-infective compound or a compound which counteracts ingrowth.

In the following, various shapes of linings or fillings or combinations of linings and fillings are described. In one embodiment, a simple lining is a thin elastomeric film that can be attached to the braided portion or to the medical device in the vicinity of the braided portion, e.g. to points on the braid mesh of the funnel. The film prevents mucosa from entering the windows between the filaments. The lining could also completely encapsulate the funnel section bound to the tip of the deployment rod forming a loose balloon around the funnel. This would require a number of drainage holes to be provided the film to facilitate drainage. Linings could also be coated with either a lubricious hydrogel coating which would reduce friction between the device and the bladder mucosa. Alternatively the lining could be coated or manufactured with a material which contains a drug which inhibits proliferation of the bladder mucosa. The lining could be fastened to the device e.g. along a rim portion of the lining or an entire inner surface of the lining could be in adhesive contact with the outer surface of the second part of the braided portion.

In one embodiment, the lining has the shape of a parachute, wherein the lining is manufactured in such a way that filaments attach the rim of the skirt to the tip of the deployment mechanism. This pulls the skirt to a collapsed position during deployment. The filaments could be made from the same material as the skirt or from a separate material that is non-elastomeric.

In another embodiment, the braided portion is protected by a lining in the form of a tube of polymeric material which is of a similar gauge or larger than the drainage tube, and which is attached to the drainage tube and the deployment rod. The tube is slit or cut along its length to enable radial expansion of the funnel with minimal resistance. The slit tube then provides periodic coverage of the funnel mesh.

In another embodiment, the lining is constituted by a foam cone, which can be located in contact with an inner surface of the braided portion and/or in contact with an outer surface of the braided portion. The foam could be bonded to the mesh and may act as a barrier to ingrowth but has the additional benefit of cushioning the filaments from the mucosa. The foam could be manufactured from a range of materials including lubricious hydrogels. The porous structure of the foam will also reduce the force needed to deploy or withdraw the mesh.

In another embodiment, the lining is constituted by a foam cylinder with a geometry being such that as the foam expands on contact with the braiding so the cylinder naturally forms a funnel. The initial cylindrical geometry is beneficial for insertion and packaging.

In another embodiment, the lining forms a cone made from a polymeric material which is attached only at the deployment rod end of the proximal end of the braided portion. The skirt collapses easily during insertion of the medical device and when the funnel is deployed, it covers a proportion of the open mesh to protect the bladder mucosa. Drainage could be further facilitated by the presence of holes in the film or foam of the skirt.

All of the above linings could be coated e.g. with a hydrogel or with a lubricious material that would reduce friction between the bladder wall and the device.

All of the above options could be presented with a soluble lining or capsule to restrain the funnel section in its smallest diameter for insertion or packaging which upon contact with liquid dissolves to free the funnel allowing deployment.

Further the funnels could be restrained in another manner which can be activated for deployment by some other means e.g. temperature, light, electricity, magnetism

The filaments of the braid could be coated in a drug delivery system which contains a drug known to reduce polypoid cystitis. Once in the bladder, the drug could diffuse from the device and prohibit the proliferation of the bladder mucosa.

As an alternative to a lining being separate from the braided portion, the lining may form part of the second part of the braided portion. As an example, the lining may be melted into or in similar way be embedded in the braiding of the second part.

To provide a slippery surface, e.g. for an improved safety of insertion of the medical device into the urethra or to reduce ingrowth, or to reduce irritation of the body tissue, at least one of the lining and the braided portion may have a hydrophilic surface e.g. comprising polyvinylpyrolidone.

To further facilitate removal from the body, the medical device could be operable between the second configuration and a third configuration by axial displacement of both the first and second parts in relation to a remainder portion of the medical device, i.e. by drawing the entire braided portion into the remainder portion of the medical device for subsequent removal of the medical device from the body lumen. During the transition from the second configuration into the third configuration, the lining could be adapted to be partly released from the second part.

Until now, it has been described that the medical device comprises an elongated tube with a braided portion with crossed filaments which form a braiding angle with the axial direction, and that the braided portion is operable between two configurations. In fact, the entire medical device could be made from a braided material, but in this case, a part of the braiding may serve as a retention section which part is therefore operable between the two configurations. The remaining part of the entirely braided device could serve as e.g. as a drainage section for draining body fluids or in general serve to establish a passage into the body. The drainage section may further comprise a matrix material, and the braiding may serve to reinforce the section so that a specific strength of the device can be obtained with a reduced wall thickness so that the cross section of the conduit can be increased to facilitate better flow of fluids trough the conduit. In the table below, it is indicated how the use of a braided drainage section can increase the cross section of the conduit in a catheter.

| | Traditional silicone catheter: | Catheter with a braided |
|---|---|---|
| Ch | cross sectional area | drainage section: cross sectional |
| size | [mm²] of conduit | area [mm²] of conduit |
| 8 | 1.3 | 2.3 |
| 10 | 3.1 | 4.2 |
| 12 | 3.8 | 7.1 |
| 14 | 4.9 | 10.8 |
| 16 | 3.8 | 14.5 |
| 18 | 8.0 | 19.6 |

In a second aspect, the invention provides a method of making a medical device comprising an elongated tube with a proximal end for insertion into a body of a living being and an opposite distal end, the tube further comprising a conduit extending in an axial direction, the tube comprising a braided portion with crossed filaments which form a braiding angle with the axial direction, the braided portion being operable between a first configuration and a second configuration wherein the braided portion is expanded to a larger size in a direction transverse to the axial direction in the second configuration than in the first configuration, and wherein passages between an outer surface of the device and the conduit are defined between the filaments said method comprising the step of reducing the cross sections of at least a part of the braided portion by application of reduction means to the braided portion.

In a third aspect, the invention provides the use of reduction means such as a lining to cover a braided portion of a medical device e.g. for reducing the risk of ingrowth of a body tissue into the device.

Though the invention has in general been described with reference to a medical device such as a catheter, the invention in a fourth aspect, further provides a an elongated tube with a proximal end and an opposite distal end, the tube further comprising a conduit extending in an axial direction, the tube comprising a braided portion with crossed filaments which form a braiding angle with the axial direction, the braided portion being operable between a first configuration and a second configuration wherein the braided portion is expanded to a larger size in a direction transverse to the axial direction in the second configuration than in the first configuration. The braiding of the tube could form passages, which in accordance with the invention are reduced in size. Such a tube could be applied in numerous non-medical applications.

Any of the features described in connection with the first aspect of the invention may also be applied in connection with the second and third and fourth aspects.

### DETAILED DESCRIPTION

In the following, the invention will be described in further details with reference to the drawing in which:

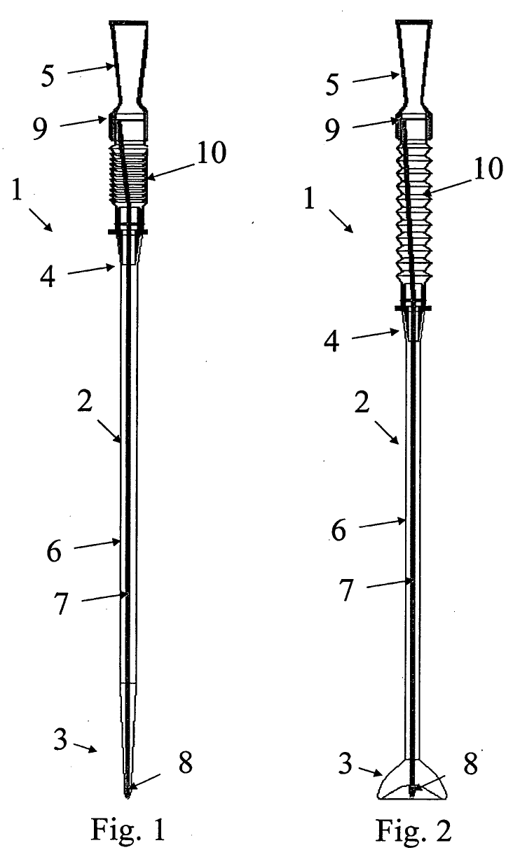

FIG. 1 illustrates a urinary catheter according to the invention, and in the first configuration,

FIG. 2 illustrates the catheter of FIG. 1 in the second configuration,

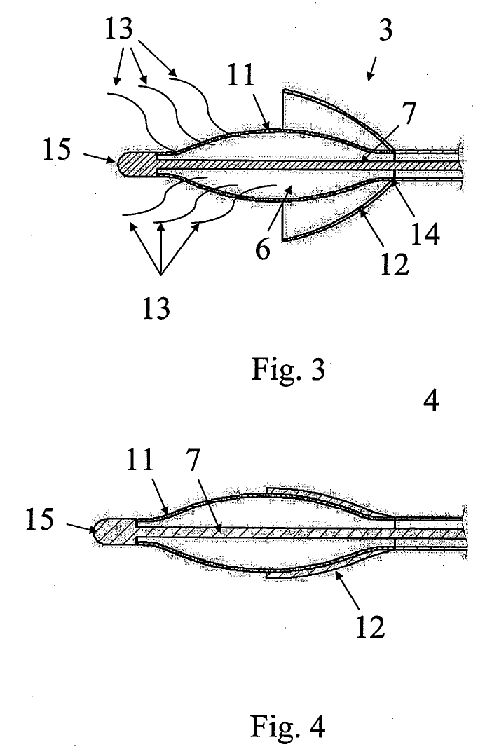

FIG. 3 illustrates enlarged views of the braided portion of the catheter in FIGS. 1 and 2 comprising a funnel shaped lining for covering a second part of the braided portion,

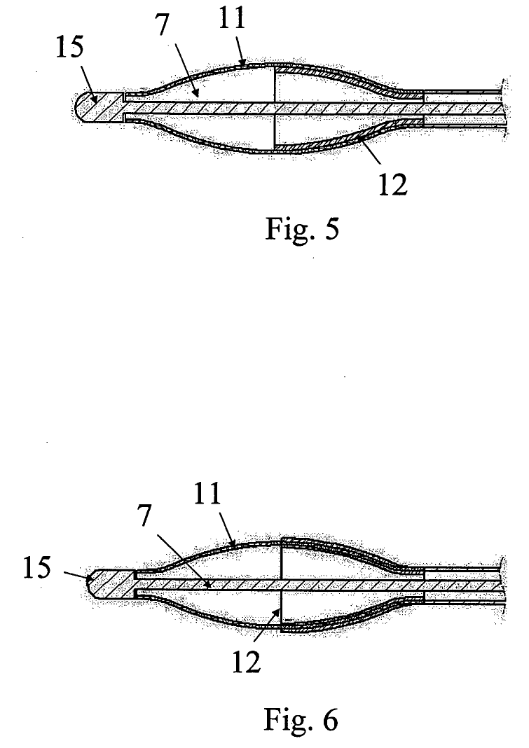

FIGS. 4-5 illustrate alternative linings,

FIG. 6 illustrates an enlarged view of the proximal catheter end, wherein the lining forms part of the braiding,

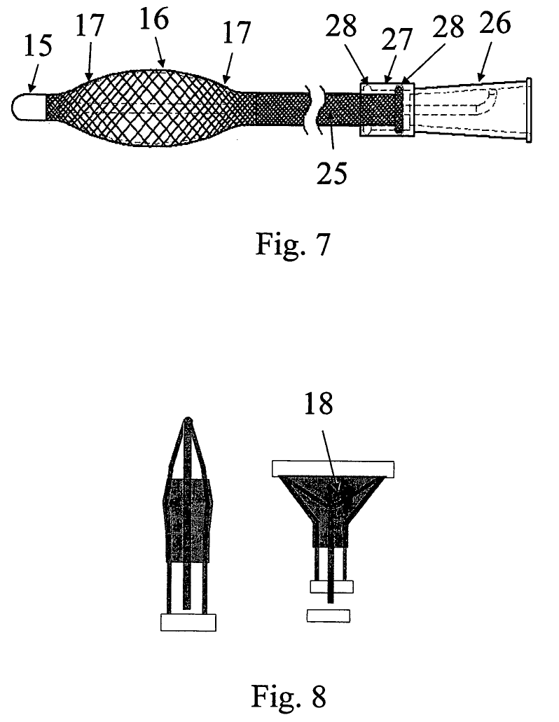

FIG. 7 illustrates an enlarged view of the braided portion wherein a first part has windows which are large relative to windows of a second part,

FIGS. 8-13 illustrate various embodiments of linings,

FIG. 14 illustrates an enlarged view of the braiding to visualise the angle of the filaments relative to each other, and

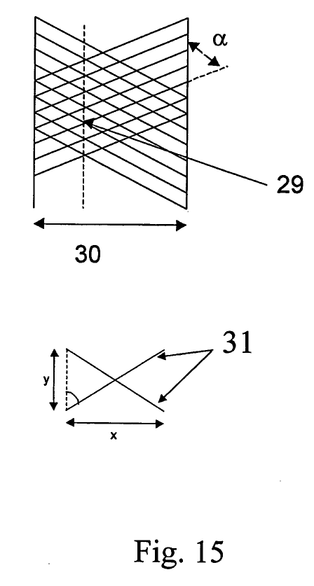

FIG. 15 shows a detailed view of single filaments of a braiding.

The device could be used for transporting fluids or substances into or out of a body, e.g. for gastro content aspiration. The device could be applied subcutaneously or through insertion of the catheter into a natural or artificial opening in the body, or the medical device could be applied for stent delivery, e.g. for placing a stent within the prostatic urethra, or in general for draining fluids from a natural or artificial body lumen, for anal insertion or for insertion into the gastrointestinal region, e.g. with the purpose of fixating a camera or surgical instruments inside the body or in general to establish a passage into the body. In the remaining part of the description, the medical device is described with reference to a catheter, and in particular to a catheter for urinary drainage, i.e. wherein the catheter is inserted into a natural or artificial urinary canal e.g. urethra, and into a bladder for draining urine.

FIGS. 1 and 2 the catheter is outlined in its full length in the first configuration i.e. collapsed for insertion into the body of a living being, c.f. FIG. 1 and in the second configuration, i.e. expanded for retention, cf. FIG. 2. As illustrated in FIG. 1, the catheter 1 comprises an elongated flexible tubular member 2 having a proximal end 3 comprising a braided portion and being formed for insertion into a body lumen, and an opposite distal end 4. In the distal end 4, the catheter has a connector 5, e.g. for connection to an elongation hose, or for connection to bag for collecting body fluid. FIG. 1 shows the catheter in its full length in a collapsed, first, configuration for insertion into the body lumen, and FIG. 2 shows the catheter in its full length in an expanded, second, configuration for retention in the body lumen. In the expanded configuration, a first part of the braided portion is drawn into the conduit whereby a funnel shaped termination of the catheter is formed. The catheter forms a conduit 6 for draining the body fluid from the proximal end to the distal end. In the conduit, the catheter has a rod 7 fastened to the proximal tip 8 and to a point 9 in the vicinity of the connector 5. To manipulate the catheter between the first and the second configurations, a bellow shaped member 10 is inserted between the two points in which the rod is fastened to the catheter. The bellow shaped member 10 allows axial displacement of the rod 7, and thus of the proximal tip 8 relative to the remaining part of the catheter. The catheter further comprises a lining 12, which is not visible in FIGS. 1 and 2, but which is illustrated in the more detailed view of FIG. 3. In the proximal end 3, a braided portion 11 forms a plurality of paths 13 for draining a body fluid from a body lumen, e.g. urine from the bladder and into the conduit 6.

In FIG. 3, the lining is attached peripherally around the catheter along an edge 14 thereof to form a parachute-like shape. When a first, proximal part of the braided portion is drawn into a second, distal, part of the braided portion, the braiding forms a funnel with a shape which matches the parachute-shape of the lining. In this configuration, the lining separates the body tissue, e.g. mucosa, from the braiding of the catheter and thus reduces ingrowth of the tissue into the catheter. The proximal tip 15 forms part of the rod 7 and is smoothly rounded facilitate safe insertion of the catheter into the body lumen.

FIGS. 4 and 5 show alternative ways of attaching the lining to the proximal end 3 of the catheter. In FIG. 4, the lining 12 is adhered to the outside of the braided portion with an adhesive. The catheter in FIG. 4 could be made by bringing the catheter to the second configuration and attaching a pre-shaped funnel shaped lining to the second part of the braided portion. The lining is fitted exactly to the funnel shaped proximal end of the catheter and, subsequently, the catheter with the affixed lining is dipped in a solvent or in a coating liquid, e.g. a hydrogel, to incorporate the lining in the braided structure. The solvent of coating liquid can be evaporated, e.g. by use of heat, and if necessary, the coating can be cured or cross-linked, e.g. by use of UV or by any other suitable cross-linking method known in the art.

In FIG. 5, the lining 12 is adhered to an inner surface of the braided portion with an adhesive, and in FIG. 6, the lining forms part of the braided structure, i.e. the lining is integrated into the meshed structure of the braiding, e.g. by reducing the size of the windows of the braiding.

In FIG. 7, the braided portion has a first intermediate part 16 with a relatively open structure with large mesh openings, and which therefore forms paths for the fluid to flow into the conduit, and second end parts 17 which has a more closed structure, and which therefore counteracts ingrowth better than the first part 16. In one embodiment, the mesh openings of the second part are completely closed, e.g. by an elastically flexible polymeric material or by a hydrophilic material. In the embodiment shown in FIG. 7, the conduit is formed in a drainage section 25, which in this embodiment is braided from filaments, i.e. almost the entire catheter is made from a braided material. FIG. 7 further shows a connector 26 wherein a slidable coupling 27 with tactile indication of its position is provided between the connector 26 and the drainage section 25. In the slidable coupling, a protrusion of the drainage section is to engage one of the two grooves 28 of the connector corresponding to one out of two predetermined positions of the tip 15 with respect to the remainder parts of the catheter. An advantage of the tactile indication is that the user may sense e.g. in the fingertips when the corrugation is brought into or out of engagement with one of the recesses. As an alternative, indication of the position of the tip could be provide visually by an indicating mark, e.g. a coloured mark.

The linings could, irrespective of the shape, be made from a soft an elastically flexible polymeric material and with a funnel shape. The funnel could be moulded and subsequently be attached adhesively to the catheter. The lining could be coated with a hydrophilic material, e.g. polyvinylpyrolidone. As disclosed, the size of the windows could increase towards an intermediate part of the braided portion.

FIGS. 8-13 show various embodiments of linings. In FIG. 8, the lining 18 is a simple lining made from a thin elastomeric film. The lining is attached to the catheter to points on the braided portion. The film prevents mucosa entering the windows between the funnel filaments. In FIG. 8, the lining covers only the second part of the braiding, i.e. the part of the braiding which could get in contact with the body tissue. The lining could, however, also encapsulate the entire braiding. In that case, openings must be made for the body fluid to drain into the conduit. The lining could be coated with either a lubricious hydrogel coating which would reduce friction between the device and the bladder mucosa. Alternatively the lining could be coated or manufactured with a material containing a drug that would inhibit proliferation of the bladder mucosa.

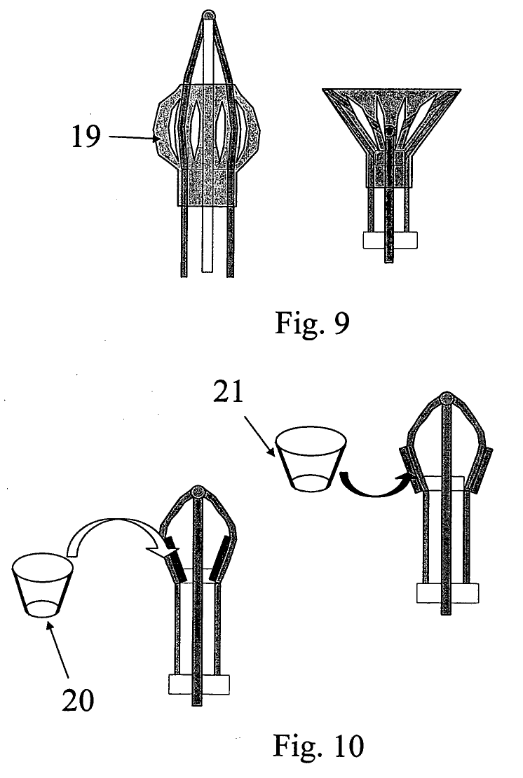

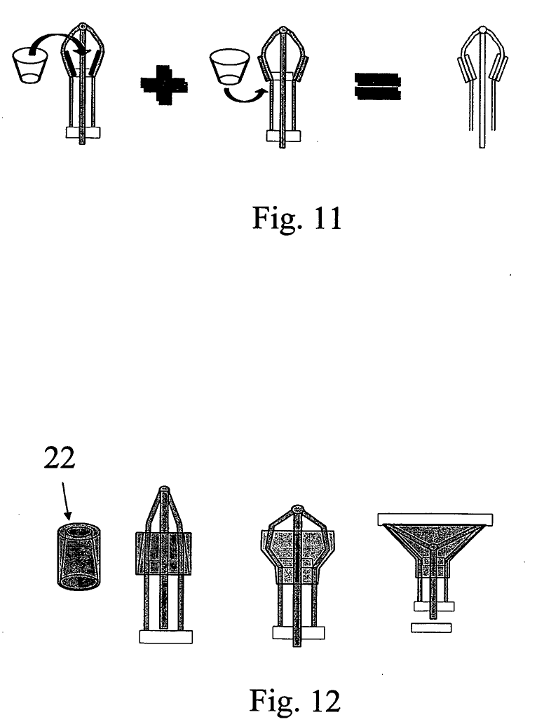

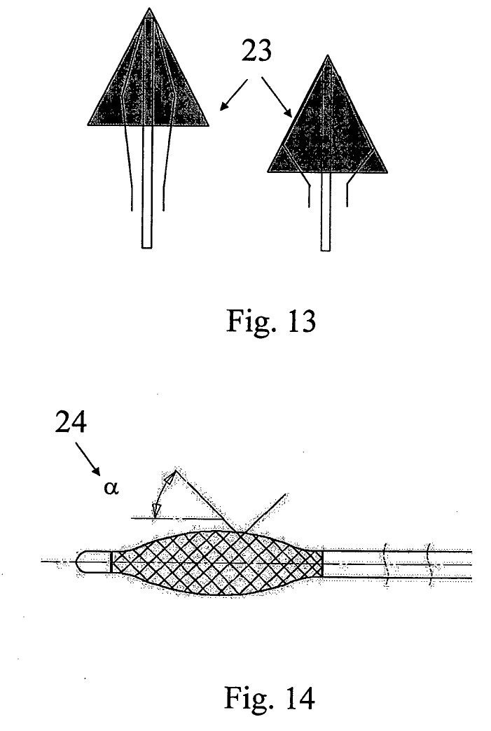

In FIG. 9, the lining 19 forms the shape of a Chinese lantern. In FIG. 10 the lining 20, 21 forms the shape of a foam cone. An inner lining 20 may be located on an inner surface of the braided portion and alternatively or in addition to the inner lining 20, an outer lining 21 may be located on an outer surface of the braided portion. FIG. 11 shows a catheter with an inner and an outer lining. In FIG. 12 foam cylinder 22 is made such that the foam expands when getting in contact with a liquid so the cylinder naturally forms a funnel. The initial cylindrical geometry is beneficial for insertion and packaging. In FIG. 13, the lining 23 is inverted and attached to a deployment rod.

FIG. 14 shows an enlarged view of the braiding of the braided portion wherein the angle, α, which the filaments of the braided portion form mutually, is indicated, c.f. numeral 24. As previously mentioned, α is important for determining maximally possible degree of radial expansion during the transition from the first configuration to the second configuration. If it is assumed that length “y” is the theoretical maximum diameter that the tube can have when expanded, and that the initial diameter “x” of the tube in its relaxed state is equal to 1, then it is possible to calculate the braid angle that is required in order to provide a funnel having a required maximum diameter using Pythagoras Theorem stating that:
Sin α=x/y

Assuming that the diameter of the tube in its relaxed state is equal to 1, then:
Sin α=1/y
making the maximum diameter of the funnel given by:

$$y = \frac{1}{Sin α}$$

At a braid angle of 15 degrees, the diameter of the expanded tube or the maximum diameter of the funnel is 3.864 times the diameter of the tube before expansion. Applied to a urinary catheter, the above theorem implies that different values of a must be applied for different sizes of catheter if funnels of equal size are desired.

With reference to FIG. 15, wherein numeral 29 indicates a braid crossover, 30 indicates the diameter of a braided tube and 31 indicates filaments, the following text describes how to calculate the braiding.

Calculation of picks per inch:

The angle of the braid is dependent on the number of yarns/filaments (N), the diameter of the rod (D) the speed of the winding table (s) and the speed of the transition of the rod through the braiding head. The speed of winding table, the speed of movement of the former rod and the number of yarns determine the number of picks per inch.

By considering a unit cell of length x and height y, it can be seen that the braid angle (α) is given by

$$tan α = \frac{x}{y} .$$

The unit cell width, x, can be expressed in terms of the machine and mandrel as follows:

$$x = \frac{2 π R}{N}$$

where R=radius of the mandrel and N=number of carriers on the braiding machine.

From the diagrams it can be seen that the number of picks per inch is inversely proportional the unit cell height, y when given in inches.

$$Number of picks per inch = \frac{1}{y} = \frac{tan a}{x}$$

Therefore, picks per inch can be expressed as:

$$ppi = \frac{N tan a}{2 π D}$$

Where D=diameter of forming mandrel, in inches.

The above equation has been modified to include the diameter of the monofilament. For very fine monofilaments, the contribution of the diameter is negligible but for larger monofilaments it must be taken into account as follows:

$$ppi = \frac{N tan a}{2 π ( D + 2 d )}$$

Where d=monofilament diameter, in inches.

Filament Spacing:

In order to consider the theoretical maximum spacing between filaments there are several factors to consider.

For a given ppi (as calculated above and measured from the centre of the filaments) the diameter of the filament (d) will determine the spacing between the extreme quadrants of the filaments.

Braiding angle α

Filament Diameter d

Perpendicular spacing between filaments P

The angular thickness of the filament

$$( b ) = \frac{d}{cos a}$$

Each angular gap between filaments for an inch

$$( g ) = \frac{ppi \times b}{ppi}$$

Perpendicular spacing between filaments (P)=g×cos α

This calculation is true for filaments with a round cross section, cross sectional geometry will have some effect on the calculated ppi value.

In the following, one example of a manufacturing process is described with reference to manufacturing of a catheter.

A forming rod which has the external diameter required for the manufacture of a catheter with the equivalent internal diameter is taken. The forming rod is partially dipped in a solution of polyurethane, where the speed of dipping withdrawal, the number of dipped layers and the total solids of the polyurethane solution dictate the film thickness deposited on the forming rod. The forming rod is coated to provide a dry film thickness of between 20 and 80 microns single wall thickness. Once the film formed on the forming rod is sufficiently dry the rod is passed through a braiding machine where, filaments are braided onto the forming rod at a given angle. The angle of the braiding is determined by the size of the forming rod, the speed of the braiding table, the axial speed of the part through the table, the number of filaments on the braiding machine and the filament size. The catheter is formed of at least 2 regions with different braid angles. The drainage section is formed from an elongated braid section with an angle in the region of 54.3 degrees to provide maximum flexibility and kink resistance. The second section is braided at a significantly lower angle to allow for the expansion of the retention means. The braided forming rod is partially dipped in a solution of polyurethane, where the speed of dipping withdrawal, the number of dipped layers and the total solids of the polyurethane solution dictate the film thickness deposited on the forming rod. The thickness of these final dips will determine the outer diameter of the catheter drainage tube. Once the polyurethane film has been dried the braided tube is removed from the forming rod by axially compressing the braided tube and sliding the forming rod from within the tube. This can be further facilitated by the use of release coatings on the forming rod or additives within the polymer dipping solution. Alternatively the forming rod can be extensible, where the forming rod decreases in diameter during axial extension, facilitating the removal of the reduced diameter rod from the internal lumen of the tube.

The connector and deployment means is then joined to the tube section, with the deployment means traversing through the internal lumen of the drainage section. The deployment means is then attached to the proximal end of the retention section in its extended configuration by thermally welding. Following the addition of the connector means an additional thin film lining is added to the catheter and may be adhered to the proximal tip, the drainage section or the retention mesh or a combination of all three. Alternatively during the dipping procedure following the braiding, the retention section may be flared/expanded and also partially dipped, utilising the surface tension of the dipping solution to form thin film webs or fillings between the filaments which when dry form a lining or filling between the filaments.

1. A medical device comprising an elongated tube with a proximal end for insertion into a body of a living being and an opposite distal end, the tube further comprising a conduit extending in an axial direction, and a braided portion being operable between a first configuration wherein it has a first size in a direction transverse to the axial direction and a second configuration wherein it has a second size in a direction transverse to the axial direction, the second size being larger than the first size.

2. A medical device according to claim 1, wherein the braided portion comprises crossed filaments which form a braiding angle with the axial direction and wherein passages between an outer surface of the device and the conduit are defined between the filaments.

3. A medical device according to claim 2, further comprising reduction means for reducing the cross sections of at least a part of the passages.

4. A medical device according to claim 1, wherein a first part of the braided portion is located inside a second part of the braided portion when the device is in the second configuration.

5. A medical device according to claim 1, wherein the conduit is dimensioned for transfer of fluids.

6. A medical device according to claim 5, and forming a catheter.

7. A medical device according to claim 1, wherein the reduction means comprises a filler material disposed between the filaments.

8. A medical device according to claim 1, wherein the reduction means comprises a lining which covers at least a surface of the second part of the braided portion.

9. A medical device according to claim 1, wherein the passages are completely sealed by the reduction means.

10. A medical device according to claim 1, wherein the lining is adhered to the second part of the braided portion.

11. A medical device according to claim 1, wherein the lining forms part of the second part of the braided portion.

12. A medical device according to claim 1, comprising a hydrophilic surface.

13. A medical device according to claim 1, comprising an opening forming path for the body liquid, the opening being located in the vicinity of a transition between the braided portion and the remaining part of the device.

14. A medical device according to claim 1, comprising an opening forming path for the body liquid, the opening being located in the vicinity of the proximal end.

15. A medical device according to claim 1, further being operable between the second configuration and a third configuration by axial displacement of the entire braided portion into the conduit of a remainder portion of the device.

16. A medical device according to claim 15, wherein the lining is adapted to be partly released from the second part upon operation of the device between the second configuration and the third configuration.

17. A medical device according to claim 1, wherein with crossed filaments form a braiding angle with the axial direction, and wherein the braid angle in at least a section of the tube, changes in the axial direction of the tube.

18. A medical device according to claim 1, further comprising a proximal tip facilitating insertion of the device, and indicating means which indicates if the tip forms a proximal end of the device.

19. A medical device according to claim 1, further comprising a proximal tip facilitating insertion of the device, and wherein the conduit and the tip are dimensioned to allow the tip to be received within the conduit.

20. A method of making a medical device comprising an elongated tube with a proximal end for insertion into a body of a living being and an opposite distal end, the tube further comprising a conduit extending in an axial direction, the tube comprising a braided portion with crossed filaments which form a braiding angle with the axial direction, the braided portion being operable between a first configuration wherein it has a first size in a direction transverse to the axial direction and a second configuration wherein it has a second size in a direction transverse to the axial direction, the second size being larger than the first size, and wherein passages between an outer surface of the device and the conduit are defined between the filaments, said method comprising the step of reducing the cross sections of at least a part of the braided portion by application of reduction means to the braided portion.

21. A method according to claim 20, wherein the application of the reduction means comprises the step of immersing at least a part of the braided portion into a composition comprising a polymeric material.

22. The use of a medical device according to claim 1 for preventing ingrowth of body tissue into the braided portion.
